(19) United States
(12) Patent Application Publication
(10) Pub. No.: US 2010/0217144 A1
(43) Pub. Date: Aug. 26, 2010

(54) DIAGNOSTIC AND PREDICTIVE SYSTEM AND METHODOLOGY USING MULTIPLE PARAMETER ELECTROCARDIOGRAPHY SUPERSCORES

(76) Inventor: Arenare Brian, Houston, TX (US)

Correspondence Address: The Law Office of Hugh R. Kress PLLC, 3120 Southwest Freeway, Suite 320, Houston, TX, 77098, US

(21) Appl. No.: 12/733,438

(22) PCT Filed: Jun. 27, 2008
(86) PCT No.: PCT/US2008/008053

§ 371 Date: Mar. 1, 2010

Related U.S. Application Data
(60) Provisional application No. 60/946,797, filed on Jun. 28, 2007.

(51) Int. Cl. A61B 5/0402

(52) U.S. Cl. 600/523

(57) ABSTRACT

A plurality of ECG Superscore formulae, created from multiple parameter ECG measurements including those from advanced ECG techniques, can be optimized using additive multivariate statistical models or pattern recognition procedures, with the results compared against a large database of ECG measurements from individuals with known cardiac conditions and/or previous cardiac events. Superscore formulae utilize multiple ECG parameters and accompanying weighting coefficients and allow data obtained from any given patient to be used in calculating that patient's ECG Superscore results. ECG Superscores have retrospectively optimized accuracy for identifying and screening individuals for underlying heart disease and/or for determining the risk of future cardiac events. They thus have greater predictive value than that of any conventional or advanced ECG measurement alone or of any non-optimized combinations of conventional or advanced ECG measurements that have been used in the past. Ongoing optimization of ECG Superscore diagnostic and predictive accuracy may be realized through the iterative adjustment of Superscore formulae based on the incorporation of data from new patients into the database and/or from longitudinal follow-up of the disease and cardiac event status of existing patients.

This application claims the benefit of U.S. Provisional Patent Application No. 60/946,797, filed on Jun. 28, 2007.

### FIELD OF THE INVENTION

The present invention relates generally to the field of electrocardiography, and more particularly to a processing system and method to analyze, combine, display, and utilize multiple electrocardiogram (ECG) parameters in a system of ECG “Superscores” that are derived from the results of three or more electrocardiographic measurements, with at least two of these measurements being advanced ECG measurements derived from at least two different advanced ECG techniques, the results of these advanced ECG techniques not being directly ascertainable or readily calculable from standard visualization or clinical inspection of the conventional ECG.

### BACKGROUND OF THE INVENTION

Conventional resting ECG is notoriously insensitive for detecting coronary artery disease (CAD) and only nominally useful in screening for cardiomyopathy (CM) and certain other cardiac disorders. Similarly, conventional exercise stress ECG is both time- and labor-consuming with suboptimal accuracy for use in population screening. Simply put, ECG-based heart disease screening methods that are presently clinically employed are inadequate to identify disease early enough and with sufficient accuracy to alert clinicians to the early onset of such disease and to help prevent the advancement of the disease. Various improvements have been made in the art to improve upon these limitations and thereby address unmet clinical needs.

Diagnosis of abnormal cardiac conditions based upon the conventional ECG has relied in the past on visible alterations in the P, QRS, and T waveforms and in the intervals between these waveforms, i.e., recognized portions of the electrocardiograph periodic signal. Deviations in various measured parameters of these waves, including their voltages, durations, gross morphology and the intervals between them, particularly deviations from a normal range or from generally accepted normal bound values, are identified as criteria to describe various abnormal or pathological cardiac conditions. There are many examples of these criteria. As one example, lengthening of the P-R interval (greater than 200 ms) is indicative of first- or second-degree atrioventricular block. Also, lengthening of the QRS interval (greater than 120 ms) is indicative of one of several possible types of ventricular conduction abnormalities. Lengthening of the QT interval (when corrected for heart rate) is indicative of one of a number of abnormalities (including electrolyte changes, drug effects, congenital syndromes or other conditions). Increases in QRS voltage in specified leads (of the typical 12-lead configuration) may be indicative of left ventricular hypertrophy (e.g., Sokolow-Lyon or Cornell voltage criteria). Other criteria from conventional ECG analysis may be indicative of other cardiac abnormalities. Many common conventional ECG abnormalities are identified clinically by a singular deviation in one type of measured conventional ECG parameter occurring in one or more leads.

At a next level of analysis, ECG abnormalities can also be identified by multiple objective or quantitative criteria specifying a particular combination of changes in two or more types of measured and clinically visualizable parameters on the conventional ECG. For example, various strictly conventional ECG scores and criteria have been demonstrated to be associated with myocardial infarction and cardiovascular mortality, such as the Minnesota code, Cardiac Infarction Index Score (CIIS) damage scores, the Simplified Selvester Score (SSS), and others, or with left ventricular hypertrophy (e.g., Romhilt-Estes score).

Furthermore, there are also examples of distinct clinical pathologies or syndromes that involve changes in two or more types of measured and clinically visualizable parameters on the conventional ECG, though quantitative criteria may be lacking in certain instances. Examples include: 1) the conventional ECG pattern of the Wolfe-Parkinson-White pre-excitation syndrome, which can include a shortened PR interval, an apparently widened QRS interval with slurred upstroke, and secondary repolarization changes reflected in ST segment and T wave changes; and 2) the conventional ECG pattern of Brugada syndrome, which can include ST segment elevation in leads V1 to V3 and various degrees of right bundle branch block (which in turn has its own well-known pattern on the conventional ECG). In general, however, conventional ECG, particularly when used in isolation, can be a very insensitive diagnostic tool. For example, a significant percentage of individuals presenting to a hospital emergency room with an actual myocardial infarction (heart attack) will have a normal 12-lead conventional ECG.

There have been a number of more advanced ECG techniques described in the art which enable more sophisticated ECG measurements. In particular, several new and advanced ECG analysis algorithms, techniques, methods and systems have recently been developed that individually advance the state of the art in some particular way. While many of these are in the public domain, others are the subject of patents or patent applications, for example, U.S. Pat. No. 7,113,820 describing a real-time, high frequency QRS electrocardiograph, U.S. Pat. No. 7,386,340 addressing an advanced ECG system for the diagnosis and monitoring of coronary artery disease, acute coronary syndromes, cardiomyopathy and other cardiac conditions, and U.S. patent application Ser. No. 11/678,839 for a multichannel system for beat-to-beat QT interval variability. The results from these advanced techniques are, by definition, not directly ascertainable or readily calculable from standard visualization or clinical inspection of the conventional ECG.

While certain individual parameters of such advanced ECG techniques may have been utilized by persons highly skilled in the art to assist with clinical decision-making in the recent past, even informally in conjunction with parameters of conventional ECG, there has not been previously disclosed a methodology for combining parameters from at least three different advanced ECG techniques, or parameters from at least two different advanced ECG techniques with one or more parameters derived from conventional ECG, in a system fashioned so as to optimize diagnostic accuracy and predictive capability (either alone or in further combination with additional non-ECG clinical data) for any number of cardiac disease conditions and/or events. Kudaiberdieva et al. [J Electrocardiology, 38(1): 17-24, 2003] have described a simple two-parameter combination of particular ECG measurements derived from two different advanced ECG techniques (as defined herein) to assess the likelihood of ventricular tachyarrhythmias in a defined clinical population (post myocardial infarction). While their method offers potential improvement for identification of certain patients at risk for this specific event, versus the even more simplified ECG methods presently employed in clinical medicine, it still uses only a limited number of ECG parameters and as such does not optimize the ability of ECG to identify such patients through the use of the more multi-parameter Superscores described in this invention. Additionally, the technique of Kudaiberdieva et al. does not provide a means for identifying any other conditions nor does it employ iteration to improve accuracy in an ongoing manner which are integral features of the present invention. The Superscores of the present invention are in contrast generalized, optimized, iterative, and extensible to an unlimited number of cardiac disease conditions as well as potential cardiac events.

In the present invention, the results from multiple ECG measurements, including from multiple advanced ECG measurements, are combined to produce ECG “Superscores” that have greater diagnostic or predictive value than that of any individual ECG measurements, or of any limited combination of ECG measurements that has been proposed or realized by others in the past. Basic premises behind the concept of ECG Superscores are first, that multichannel ECG recordings contain sufficiently detailed information to allow for detection of most cardiac pathology, and second, that while there may be a multiplicity of advanced ECG parameter patterns that point to any given categorical disease process or combination of disease processes, ultimately, the most crucial or useful of these patterns are ascertainable from retrospective population studies and can be codified (as well as continuously improved and reiterated) for subsequent use in evaluating new patients. Advanced ECG measurements utilized in ECG Superscores can include: 1) Signal averaging of P, QRS and T waveforms, with or without accompanying bandpass or other filtering, to derive unfiltered or filtered parameters of waveform amplitudes, durations, axes, angles, slopes and velocities; 2) Decomposition of P, QRS, and T waveforms, including of signal averaged P, QRS and T waveforms, by techniques such as principal component analysis, independent component analysis, and singular value decomposition, to derive not only individual eigenvalues and eigenvectors for the P, QRS and T waveforms separately or in combination, but also any number of parameters that constitute mathematical relationships between the eigenvalues and eigenvectors of these waveforms; 3) Studies of spatial (including 3-dimensional) parameters of the P, QRS and T waveforms, including of signal-averaged P, QRS and T waveforms, wherein there is a reliance upon reconstruction of the 3-dimensional Frank or other set of 3-dimensional (“X, Y and Z”) channels or vectors from incoming data that does not natively provide such a 3-dimensional representation. Parameters that can be derived from reconstructed 3-dimensional channel- or vector-related information include, for example: lead-specific or vector-specific (i.e., spatial) magnitudes, durations, orientations, angles and velocities of unfiltered or filtered P, QRS and T waveforms, or of the spatial ventricular gradient; the spatial angles between the unfiltered or filtered spatial P, QRS and T waveforms; and the beat-to-beat variabilities of any of the above components; and 4) Beat-to-beat variability studies of the P, QRS and T waveforms or of the time intervals between or amongst them, wherein the raw ECG data emanates from any type of ECG channel system. Such parameters include, for example, parameters of beat-to-beat RR, PP, PR, PQ, QRS, QT, Q-Tpeak, RT, R-Tpeak, JT, or J-Tpeak interval variability, beat-to-beat variabilities of the unfiltered or filtered P, QRS or T waveform amplitudes or of ST segment amplitudes, and other advanced parameters of variability including, for example, “unexplained” interval variability, wherein that part of the given interval's (e.g., QT interval's) variability that can be readily explained by RR interval variability and/or by other extrinsic factors ascertainable from the advanced ECG (such as respiration-related changes in voltage amplitudes, QRS-T angles and other factors) is eliminated from total interval variability, thus isolating the variability's “unexplained” portion, as well as indices of ECG dipole variability utilizing a set of real or derived X, Y, Z dipole vectors optimally matching the eigenvectors of a singular value decomposition transformation matrix.

These advanced ECG techniques can be used simultaneously and can be obtained using standard electrode and lead configurations. Typically, best results with these techniques are obtained when a plurality of beats (such as 100 or more) are processed and analyzed, though they also work with ECG recordings of shorter duration.

The advanced measurements described herein provide examples only, and should not be construed to provide an exhaustive list of all possible advanced ECG measurements that may be used in ECG Superscores. In general, it is customary to consider any ECG parameter that is not directly ascertainable on or readily calculable from the conventional ECG, and that usually requires additional signal processing in software for its accurate and/or clinically timely derivation, as an “advanced” ECG parameter, in opposition to a “conventional” ECG parameter, which on the contrary is easily recognizable on, or ascertainable from, a conventional ECG tracing, for example by using a physical calipers (or electronic calipers, in the case of computerized ECG recordings). It is acknowledged that there are rare “gray areas” wherein it might be reasonably disputed as to whether a particular parameter should be considered as “conventional” or “advanced”. However, practically speaking, it is useful to define an “advanced” ECG parameter as one that a majority of medical practitioners—including cardiologists and other experienced readers of ECGs—would usually not attempt to manually determine (nor feel confident in “over-reading”, in the case of the practitioner disagreeing with an automatically provided result on the ECG) during the course of typical clinical practice.

Each of these advanced algorithms and techniques may individually provide, for any given patient, potentially clinically useful information about heart disease conditions, the risk of developing such conditions, and/or the risk of certain arrhythmias or other cardiovascular events, including sudden death. Whether applied individually in isolation or together, these techniques have varying degrees of potential clinical utility for diagnosis and/or prognosis, and may offer tangible improvements in accuracy over other strictly conventional ECG methods for determining the presence or absence of various disease conditions and/or the presence of altered disease or event risk. Moreover, changes over time in the results or findings of any of these tests (or others like them) can provide important contributions to disease management, including the choice of medical and procedural interventions, and follow up care. However there remains a need for a methodology and system that optimally combines and integrates the results of multiple parameters measured by ECG tests in order to provide more effective noninvasive clinical diagnostic ECG assessments and to more appropriately guide medical therapy and intervention. The present invention is directed to filling this need in the art by offering a methodology and system that not only produces but also combines the results of several ECG techniques in such a fashion as to realize increased clinical usefulness and accuracy within the field of ECG.

### SUMMARY OF THE INVENTION

In the present invention, a system and a method are disclosed in which the benefits of performing multiple advanced ECG techniques along with conventional ECG techniques are yet furthered through deriving and utilizing specific optimized combinations of measurements from such ECG techniques so as to better detect and screen for specific types of heart disease and to better identify the risk of specific types of cardiac events. This improved detection and screening process results in the stratification of the probability of the presence and/or risk of any given cardiac disease or the risk of any given cardiac event for an individual patient.

The present invention offers a methodology for combining a plurality of ECG measurements to: 1) improve the noninvasive ECG detection of a variety of cardiovascular diseases, such as CAD, acute coronary syndromes (ACS), ischemic and non-ischemic cardiomyopathies (CMs), ventricular hypertrophy, ion channelopathies, and many other conditions, and to 2) improve the noninvasive ECG prediction of the risk of cardiac events such as arrhythmias and sudden cardiac death. Such ECG measurements may include (but are not limited to) those described above. ECG Superscores are derived utilizing the methodology of the present invention in combining multiple ECG parameters from such advanced and also from conventional ECG methods. For cardiac disease in general, and for specific cardiac disease and event categories, the methodology may be utilized to construct one or more Superscore formulae for identifying the given disease and/or predicting the given event.

Optimization of diagnostic and/or predictive accuracy of ECG Superscores is an integral element of the methodology. A database is utilized that incorporates various individual and aggregate patient data, including, for example, known cardiac conditions and risk factors, results of previous “gold standard” imaging and/or invasive studies such as cardiac catheterization, all ECG records as well as any known outcome information such as cardiac events. Optimized disease- and/or event-specific ECG Superscores are formulated by using relevant elements of the database to retrospectively maximize the Superscores' areas under receiver operating characteristic curves against typical “gold standard” clinical information. This is accomplished through the use of ECG parameter selection procedures, including, for example, branch-and-bound, and/or traditional (forward/backward), nested or otherwise optimized stepwise selection procedures. ECG parameter selection for Superscores takes place within the context of constructing an additive multivariate statistical or other model using either traditional statistical (e.g., logistic, linear) or pattern recognition-type techniques (e.g., support vector machine models, neural network models, recursive partitioning models, classification and regression tree models, linear, quadratic, logistic, and Kth nearest neighbor discriminant models, etc.). In datasets containing several hundreds of ˜5 min resting ECGs, several Superscores employing such models are presently more than 90% accurate in identifying both obstructive CAD and CM. Clinical data and advanced and conventional ECG data for any new or existing patient may be iteratively added to the database, allowing ongoing refinement of Superscore formulae and improved accuracy as these data are added, thereby helping to improve the accuracy of Superscores applied to any future patient's ECG data.

Key parameters utilized in any given Superscore tailored to any given cardiac disease category and/or to cardiac event risk may vary, depending on the pathologic condition of interest and the particular statistical or pattern recognition technique utilized, and the amount of previous optimization that has been performed. For example, in the clinical situation of a relatively readily ascertainable or diagnosable condition, such as cardiomyopathies wherein the echocardiographic ejection fraction is proven to be less than approximately 40%, ECG Superscores may only need to contain as few as three or four individual ECG parameters. However, most Superscores include many more individual parameters and draw upon the majority of advanced ECG techniques described above. Standard- or high-fidelity ECG testing employing these multiple parameter Superscores offers a rapid and inexpensive new tool for the early diagnosis, screening and monitoring of heart disease.

Several of the advanced ECG parameters that are used in several of the ECG Superscores pertaining to the present invention are described in greater detail below. It should be emphasized, however, that these do not provide an exhaustive list, in terms of the spirit of the invention.

The present invention addresses needs in the art by providing a method and system that readily combines multiple ECG parameter measurements, obtained during one or more ECG data collection sessions, into a clinically meaningful integrated form, denoted as an ECG Superscore, that improves diagnostic and/or predictive accuracy over all present ECG techniques known in the art. The invention also provides a system for a display and a method of displaying such aspects as Superscore results.

The utility of the present invention has been recently assessed in a clinical research context in an as yet unpublished scientific investigation entitled “Construction and Use of Resting 12-Lead High Fidelity ECG “SuperScores” for Detection of Heart Disease” by the inventor and other co-authors. In this study of nearly 700 individuals, a 14-component resting multivariate 12-lead ECG Superscore was found to have 97% accuracy for detecting the presence versus absence of heart disease, significantly greater than the optimal accuracy for pooled conventional 12-lead ECG criteria alone. Clinical use of ECG Superscores may potentially streamline certain aspects of medical decision-making related to heart disease, as well as improve the overall cost effectiveness of medical care. Just as a number of ECG “signatures” can identify particular diseases on the conventional ECG, so too may several otherwise undiagnosed cardiac diseases become more readily recognizable through pattern recognition during ECG Superscoring.

Simply put, ECG Superscores combine and integrate measurements obtained from multiple advanced ECG techniques, and also when appropriate from conventional ECG techniques, into a more clinically meaningful, useful and practically relevant form. The invention includes a number of features that are neither shown nor suggested in the art, including a new means by which to utilize a noninvasive ECG test to, as we have found, accurately predict the results of invasive tests such as coronary artery catheterization, or to successfully predict the presence or absence of clinically meaningful coronary artery disease with >90% accuracy or of cardiomyopathy with >95% accuracy.

These and other objects and advantages of the present invention will be apparent to those of skill in the art from a review of the following detailed description.

### BRIEF DESCRIPTION OF THE DRAWINGS

FIG. 1 is a schematic diagram of the overall system of this invention.

FIG. 2 is a diagram showing the steps in the construction and use of ECG Superscores.

FIG. 3 is an example decision tree graphic derived from recursive partitioning for improved detection of ischemic heart disease based on advanced plus conventional ECG

FIG. 4 is an example leaf report graphic derived from recursive partitioning for improved detection of ischemic heard disease based on advanced plus conventional ECG.

FIG. 5 is an example graphic of a neural network model for diagnosis of ischemic heart disease that employs the same parameters as shown in FIGS. 3 and 4.

FIG. 6 shows examples of a methodological model to identify disease based on multiple discriminant analysis using advanced plus conventional ECG.

FIGS. 7A and 7B show examples of the methodological model to identify disease based on specific discriminant analysis using advanced plus conventional ECG.

FIG. 8 is a sample monitor display or printed report of ECG Superscores.

### DETAILED DESCRIPTION OF PREFERRED EMBODIMENTS

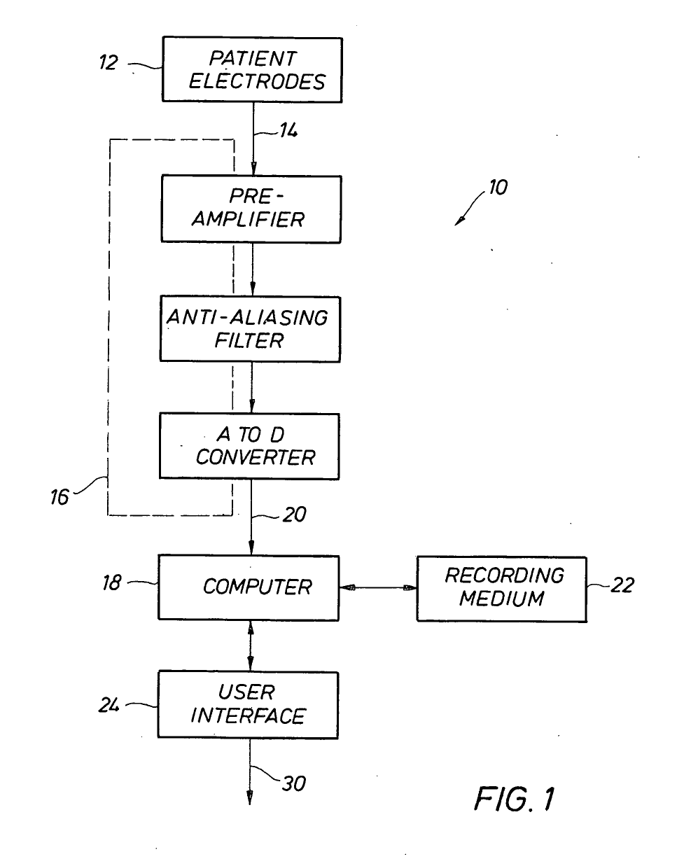

FIG. 1 shows a simplified, functional, block diagram of a multichannel electrocardiographic monitoring and data storage system, 10 adapted to carry out the present invention. The invention monitors the cardiac function of a patient with a plurality of patient electrodes 12. The electrodes 12, when attached to an appropriate lead wire cable 14, provide measurements of cardiac electrical function at or between various contact points on the skin of a patient in the conventional manner. For example, in the conventional 12-lead configuration, ten electrodes placed upon the skin of the patient in the conventional configuration provide eight channels of incoming analog data.

A console 16 conditions and digitizes the incoming analog data from the cable 14 and provides the digitized signal to a computer 18 by way of a communications channel 20, which may preferably be a conventional cable, a network connection, or a wireless communication channel by radio frequency wave. In a preferred embodiment, various functions of the signal acquisition and process are carried on by multiple processors. The computer is programmed to display the ECG signals in real time, although the ECG signals may also be stored on a digital recording medium 22 for later analyses. The computer is programmed to automatically detect the RR, PR, QRS, QT and other intervals, on a beat-to-beat basis, and to compare those detected intervals to continuously updated templates, including signal averaged templates, also developed by the computer. The computer can moreover translate the digital signals into twelve lead data, and/or into Frank or other X, Y, Z lead data, or any subset thereof.

The computer 18 is coupled to a user interface 24 which preferably includes direct or indirect connections to other devices such as a mouse, keyboard, and/or touch screen and/or printer. The user interface further includes a monitor for user controllable graphical and/or numerical display of the results of ECG measurements, including the components, coefficients and results of ECG Superscores which are features of the present invention.

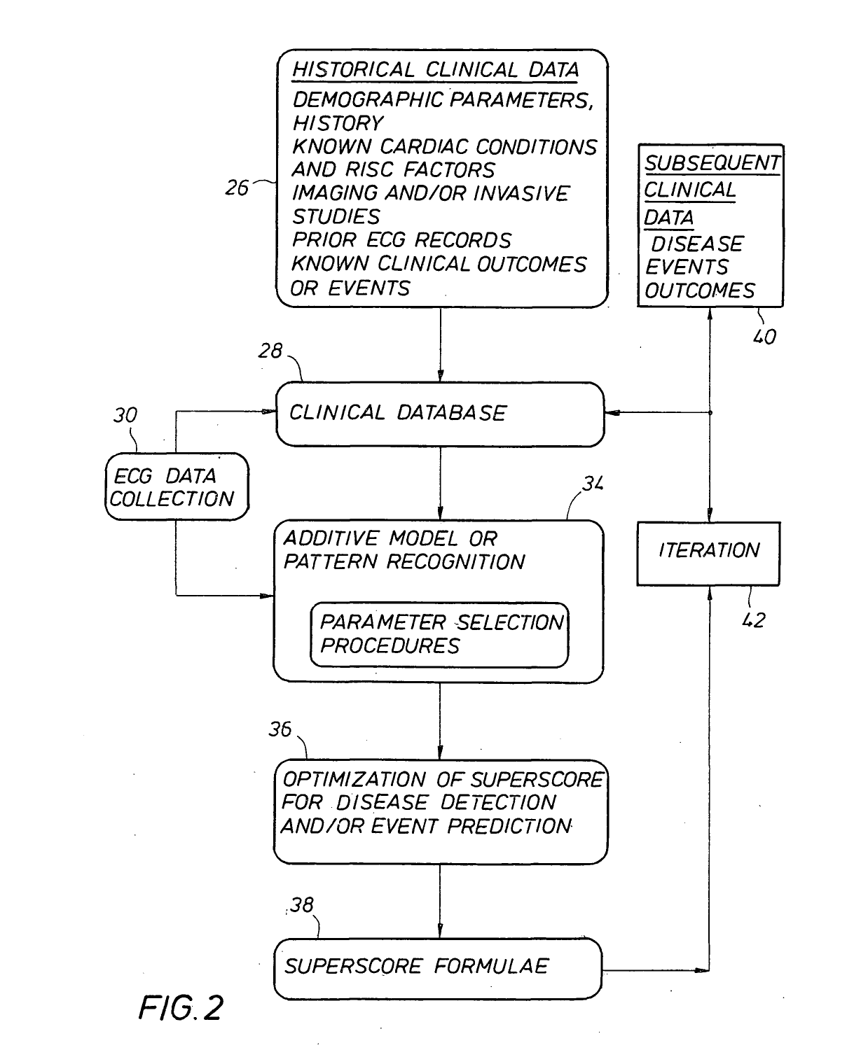

FIG. 2 delineates the steps involved in the construction and use of ECG Superscores. In one preferred embodiment, historical clinical data 26 may comprise individual and aggregate patient information, including demographic parameters such as age and gender, medical history, known disease status and risk factors, the results of cardiac catheterization or other imaging or invasive studies, known laboratory results, known prior ECG results, and any known outcome information such as cardiac events, etc. This information is maintained in a clinical database 28 along with recordings from ECG data collection 30 (See FIG. 1). One or more multichannel ECG recordings, ideally of high fidelity, are obtained from a resting, supine patient, with a minimum number of accepted beats obtained, usually requiring from two to five or more minutes. Collected ECG data are then used in subsequent parameter selection procedures 32, based upon information in the database. Parameter selection occurs in the context of an additive multivariate model or pattern recognition technique 34. Using information from the database 28, the selected parameters are combined optimally in an optimization engine 36 to construct the final Superscore formulae 38. The database 28, and the Superscores ultimately derived using it, offer a means for any individual patient's overall results to be compared and contrasted with those of known populations of diseased and healthy individuals whose data also reside in the database. Additional and subsequent clinical and ECG data 40 may be used to progressively and repeatedly re-optimize Superscore formulae through a process of iteration 42. Over time, with an increasing size of the database, the accuracy of Superscores in determining disease and predicting events is thereby likely to even further increase from that following the original optimization.

An ECG Superscore may appear, in a most simplified linear form, as:

Superscore (SS)=[a1]+[b2]+[xN]+ . . .

Wherein 1, 2 . . . N represent the results of the ECG techniques that are the components of the given ECG Superscore and wherein a, b, . . . x represent the population statistics-derived numerical weights for each of those respective components. As an example, logistic regression analysis can be used to estimate the probability of a new patient being a member of a particular disease or event-risk group based strictly on his/her ECG variables. Classification of patients can be made on the basis of whether or not the predicted probability of being in a disease or event-risk group is greater than or less than, for example, 0.5. In terms of a specific vector x, of particular ECG measurements, the classification rule is equivalent to deciding “Disease Type A” (or “Event-risk Type A”) if a linear combination of the measurements, say b'x exceeded a threshold c, where b=(b1, b2, . . . , bn)′, the vector of coefficients and the constant c being obtained from the regression analysis. The criterion b'x−c is in this case the same as the ECG Superscore. Through the use of parameter selection procedures, including, for example, branch and bound, and/or traditional (forward/backward), nested, or otherwise optimized stepwise procedures, promising x-vectors, i.e., candidate sets of parameters x for inclusion in Superscores, can be identified. The best candidates can then be subjected to validation by bootstrap analyses in which for each fixed x, the data can be iteratively resampled any number of times and the coefficients (bi) re-estimated. The bootstrap analyses can reveal those candidate sets of ECG parameters which can or cannot be reliably used to define a rule for classifying subsequent unknown single cases. For example, if too many parameters are included in x, the resulting coefficients may vary wildly over the bootstrap samples, indicating that a classification rule based on that x would be potentially unreliable. In addition to stability of the coefficients, the coefficients for each individual parameter should ideally have their anticipated (as well as unvarying) negative or positive signs over all of the bootstrap samples. If this is not the case for all (or nearly all) of the bootstrap samples, then an associated Superscore may not be considered valid and might be discarded.

A disease or event specific ECG Superscore (SS-DDDn) may alternatively take a variety of non-linear forms, and generally, as:

Superscore.(SS-DDDn)=f[advanced ECG parameter 1]+f[advanced ECG parameter 2] . . . +f[advanced ECG parameter N]+f[conventional ECG parameter N],

where at least one f is a non-linear function.

An example of one four-component ECG Superscore for coronary artery disease (CAD) (simplified here for the purposes of illustration) is as follows:

Superscore SS-CAD1=(High Frequency QRS ECG Reduced Amplitude Zone Score/6)+0.1*(Principal Component Analysis ratio of T wave)+4*(QT Variability Index)−2*(In low frequency power of RR interval variability)

where: High Frequency QRS ECG Reduced Amplitude Zone Score, Principal Component Analysis ratio of T wave, QT Variability Index and low frequency power of RR interval variability are all parameters from the advanced ECG (see below).

Of course, in reality, the given coefficient weightings for an optimized Superscore are typically not whole numbers as shown in the above example, but rather extend out several decimal places, such that the second and third weights above might actually be closer to “0.1013489” and to “4.10768447”, respectively, rather than 0.1 and 4, respectively.

Superscores may be optimized for specific disease and/or event categories, including but not limited to: CAD, ACS, CM (both generally and including separately ischemic, non-ischemic and hypertrophic), ventricular hypertrophy, Chagas' Disease, ion channelopathies, right ventricular dysplasia, and the risk of events such as sudden cardiac death (SCD) or of atrial and ventricular fibrillations and tachycardias. For individual specific disease and event categories (e.g., CAD, SCD, etc.) there may be any number of ECG Superscores (SS) for the given category (i.e., SS-CAD1, SS-CAD2, . . . SS-CADn; SS-SCD1, SS-SCD2, . . . SS-SCDn) which are optimized for accuracy by combining the specific terms from multiple ECG techniques. By parameter selection and weighting adjustment of the variables in combination, the Superscores are optimized against a large retrospective database of ECG recordings from patients with and without the specific disease category and/or event who have also had other, “more definitive” and expensive medical tests (invasive and noninvasive) such as, for example, perfusion imaging, stress and non-stress echocardiography, angiography, computerized tomography and magnetic resonance imaging. Thereby, a specific Superscore is made to have maximal accuracy for identification of individuals in the given disease or event risk category, based upon such retrospective data. There are generally at least two advanced ECG parameters that must be incorporated into a given Superscore to ensure reasonably high accuracy for the given disease or event category. Moreover Superscores may be expressed not only as probabilities but also as absolute or normalized scores with easily recognizable cut-offs. For example Superscores can be readily transformed so that “0” (or “10”, “100”, etc.) represents a cut-off point, with <0 (or <10 or <100, etc.) indicating low severity (and/or low risk) and >0 (or >10 or >100, etc.) indicating high severity (and/or high risk), etc.

In the presently preferred embodiment of the invention, ECG Superscores are derived from one or more additive models, support vector machines, discriminant analyses, neural networks, recursive partitioning analyses, or classification and regression tree analyses, many of these techniques being referred to as pattern recognition techniques by those experienced in the art. The Superscores are then used to predict, offline or in real time if desired: 1) the presence or absence of any given cardiac disease in the given patient; and/or 2) the severity of any given cardiac disease in the given patient, if cardiac disease is already known to be present; and/or 3) the risk of a cardiovascular event in the given patient; and/or 4) the risk of cardiovascular mortality in the given patient. In contradistinction to other pre-existing clinical “metascores” (such as the Thrombolysis in Myocardial Infarction or “TIMI” risk score, etc.) that usually rely heavily upon clinical information such as patient age, medication use, clinical history, the number of traditional risk factors present for CAD, etc., the application of Superscores in the presently preferred embodiment does not depend upon knowing any piece of clinical or demographic information from a new patient beyond the results of his/her ECG. In their practical application in the presently preferred embodiment, the Superscores either: 1) combine the results derived strictly from three or more advanced ECG techniques; or 2) combine the results from one or more conventional ECG techniques with those from two or more advanced ECG techniques. However, it is easy to envision that in the future, and within the spirit of the present invention, that clinical and demographic factors such as age, gender, blood pressure, other risk factors, laboratory results, etc., might be adjoined to (or made additional constituents of) ECG Superscores in an effort to further enhance accuracy, this being an aspect of the present invention.

In still another aspect of the invention, the Superscores are iteratively re-optimized or “fine tuned” through one or more of at least three means: 1) continued retrospective analysis of patient data comparing conventional and advanced ECG results to the results from other, “more definitive” and expensive medical tests (invasive and noninvasive) such as, for example, perfusion imaging, stress and non-stress echocardiography, angiography, computerized tomography and magnetic resonance imaging; and 2) forward (prospective and longitudinal) analysis of ECG data from patients who have not yet had one of these more definitive and expensive tests but yet who later go on to have one or more of them after they have had initial EGG Superscoring; and 3) the addition (or substitution) of the results from promising new ECG parameters into the ECG Superscores when such promising new parameters are discovered. At the present time, the practical usefulness of the ECG Superscores emanates from possession and study of large existing databases of ECG data derived from persons who have known disease and who are known to be free of disease, but with this practical usefulness also continually improving in an iterative fashion, as more and more advanced ECG data from more and more patients (or from new ECG parameters) are added to the existing large database.

The ECG Superscores have typically been obtained from 12-lead resting ECG recordings of several minutes duration (typically about 5 minutes or about 300 heart beats). However, as long as advanced ECG software is utilized, many Superscores can also be obtained from a short-duration (8 to 10 second) 12-lead ECG, or from a similarly short duration “limb lead only” or other ECG configurations, for example from an exercise ECG, or from a prolonged ECG of any duration, for example during Holter monitoring or bedside monitoring. Similarly, Superscores can also be derived from Frank or other “orthogonal lead” ECG configurations, including the so-called “EASI” leads, reduced lead sets, etc.

Moreover, any duration of ECG monitoring that employs advanced software can also utilize real-time ECG Superscoring and make note of any changes in Superscore results, such as, for example, during a medical or procedural intervention. The change in ECG Superscore results over time in any given individual is also of note as a potential indicator of disease progression, remission, or stability.

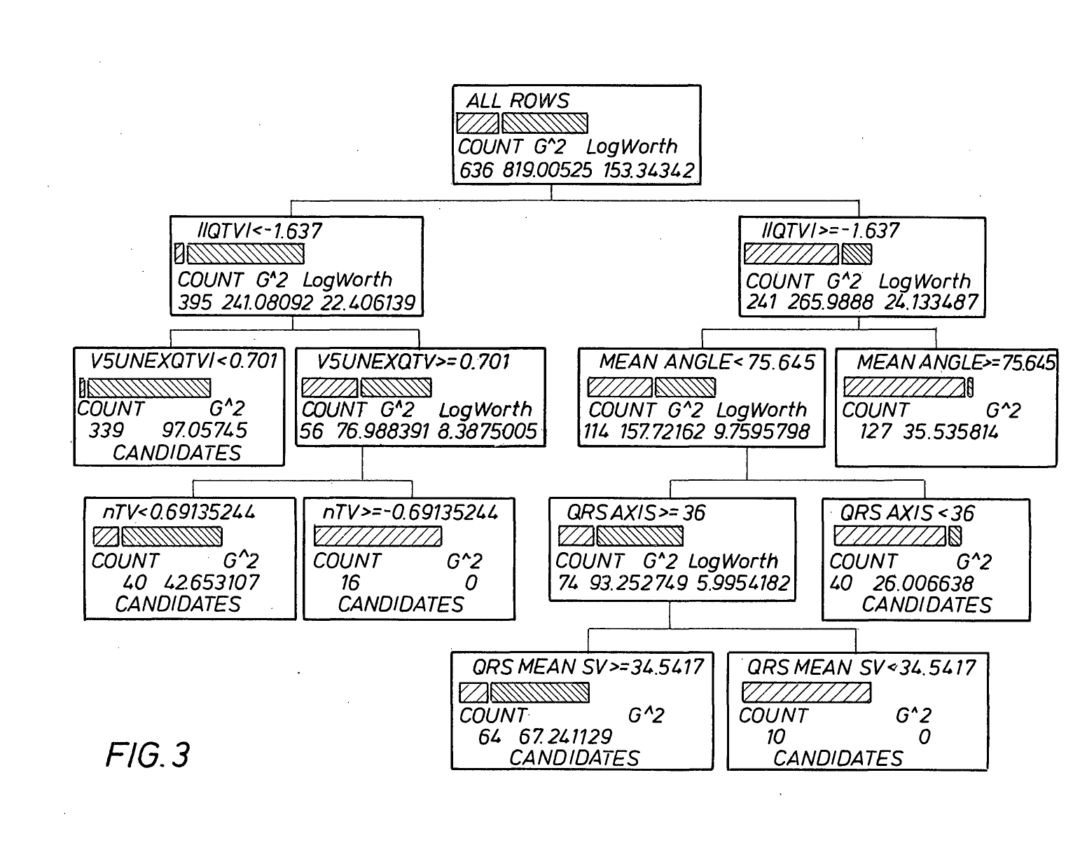

One or more additive models or pattern recognition techniques may be utilized for parameter selection and Superscore optimization. FIG. 3 for example shows a decision tree (first six steps only) derived from multivariable recursive partitioning analysis that results in improved detection of ischemic heart disease based on the incorporation of results from parameters of both advanced and conventional ECG. Recursive partitioning is a method for the multivariable analysis of medical diagnostic tests in which a decision tree is created that strives to correctly classify based on a dichotomous dependent variable, in this case, the presence or absence of ischemic heart disease. In FIG. 3, IIQTVI is the index of beat-to-beat QT variability in lead II, in specialized units; V5UnexQTVI is the index of “unexpected” QT variability in lead V5, in specialized units; nTV is the normalized 3-dimensional T wave volume, a measure of T-wave complexity derived from singular value decomposition of the T wave, in units of percent; Mean Angle is the spatial mean QRS-T angle in units of degrees; QRS axis is the axis of the QRS complex in the conventional ECG frontal plane, in units of degrees; and QRS Mean SV is the mean spatial velocity of the signal-averaged spatial QRS wave, in units of millivolts per second.

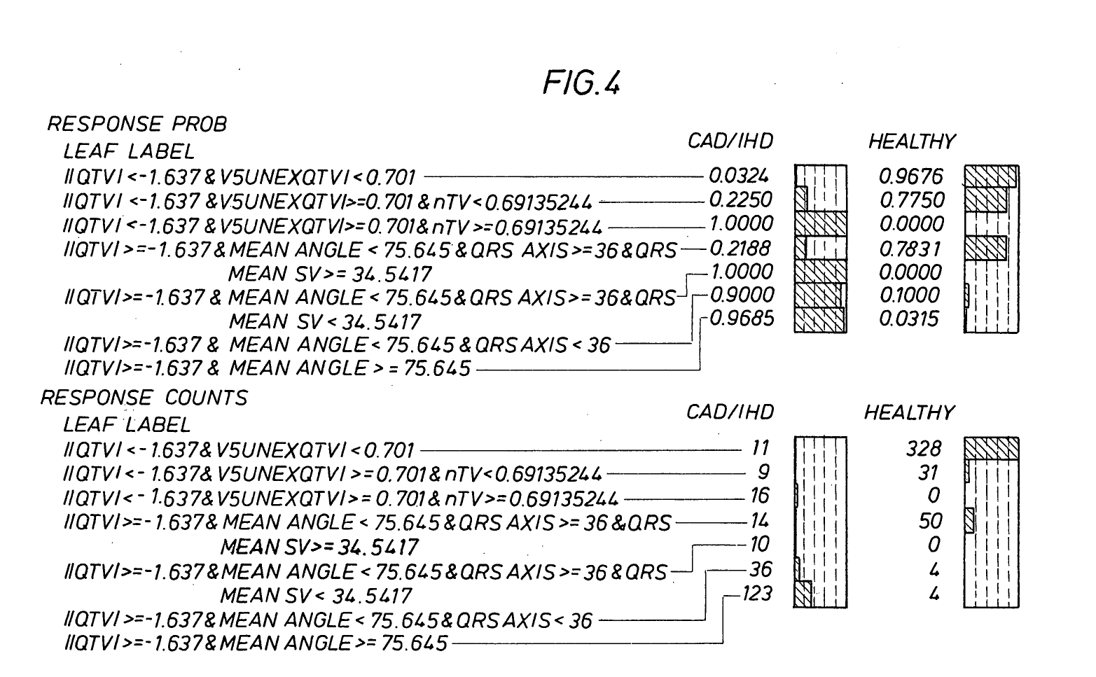

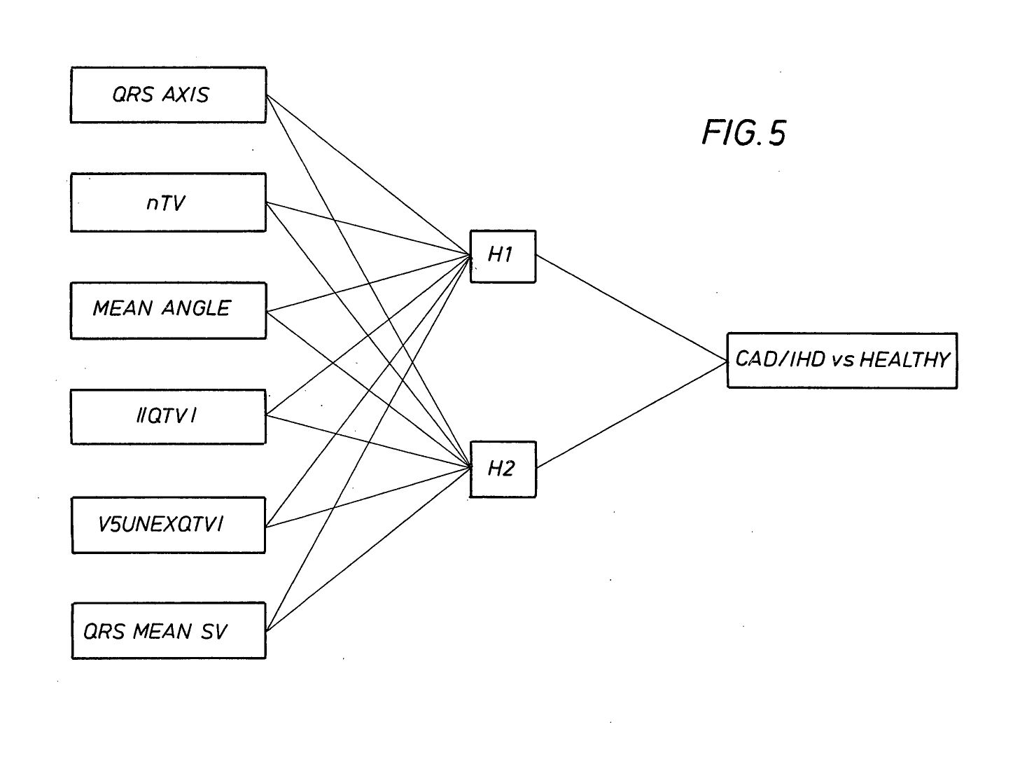

FIG. 4 illustrates an example leaf report graphic for the six-stepped recursive partitioning of FIG. 3. For each leaf (node without child nodes in the decision tree structure) the probability of ischemic heart disease and patient count are identified numerically and graphically. An example of another pattern recognition technique, in this case a neural network model, applied to the formulation of Superscores (again, for ischemic heart disease) is shown in FIG. 5, which depicts a schematic neural network diagram that employs the same parameters as shown in FIGS. 3 and 4, and where H1 and H2 are (in this case) two “hidden nodes” of the neural network. An artificial neural network involves a network of simple processing elements (artificial neurons) which can exhibit complex global behavior, determined by the connections between the processing elements and element parameters. In a neural network model, simple nodes are connected together to form a network of nodes—hence the term “neural network”. While a neural network does not have to be adaptive per se, its practical use comes with algorithms designed to alter the strength (weights) of the connections in the network to produce a desired signal flow.

Discriminant analysis is a pattern recognition technique that utilizes and combines those variables that, together, best discriminate between two or more naturally occurring groups. By canonical analysis, multiple function discriminant analysis can automatically determine some optimal combination of independent or orthogonal variables so that the first function provides the most overall discrimination between groups, the second provides second most, and so on. Discriminant analysis as applied to advanced ECG also provides an intuitive graphical means of aiding interpretation of quantitative data. Types of discriminant models can include, for example, linear, quadratic, logistic, and Kth nearest neighbor discriminant models, or a discriminant model based on a support vector machine.

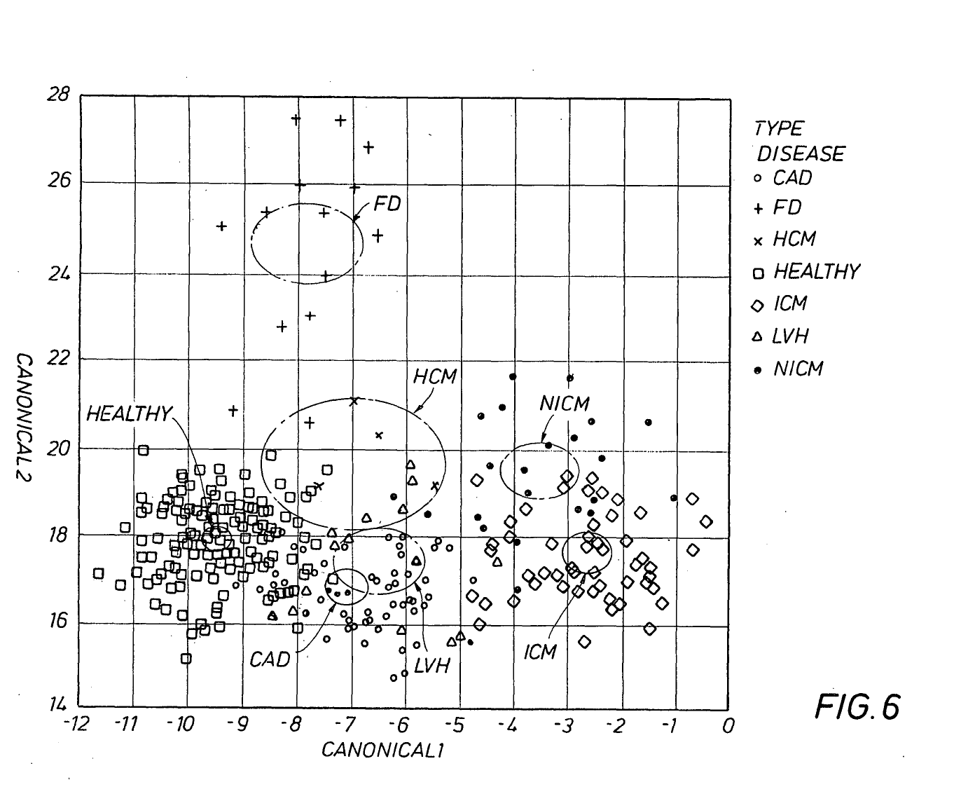

FIG. 6. shows an example of another aspect of the present methodology which employs a multiple discriminant analysis using advanced plus conventional ECG to identify patients whose ECG data are suggestive of one (or more) of a variety of cardiac diseases simultaneously. Legend: CAD=Coronary Artery Disease. HCM=Hypertrophic Cardiomyopathy ICM=Ischemic Cardiomyopathy. NICM=Non-Ischemic Cardiomyopathy. FD=familial dysautonomia (a rare autosomal recessive disease occurring principally in young Ashkenazi Jews). In the graphic each individual is represented by a unique symbol and the analysis classifies each individual with the condition in a 2-dimensional locus of points. It should be noted that less than 5% of individuals are misclassified into a condition that is other than their own. This is very impressive given the number of conditions that must be discriminated from one another. Such graphics can also be displayed and manipulated in 3 dimensions (rather than 2 dimensions as shown) in order to provide a visually improved discrimination.

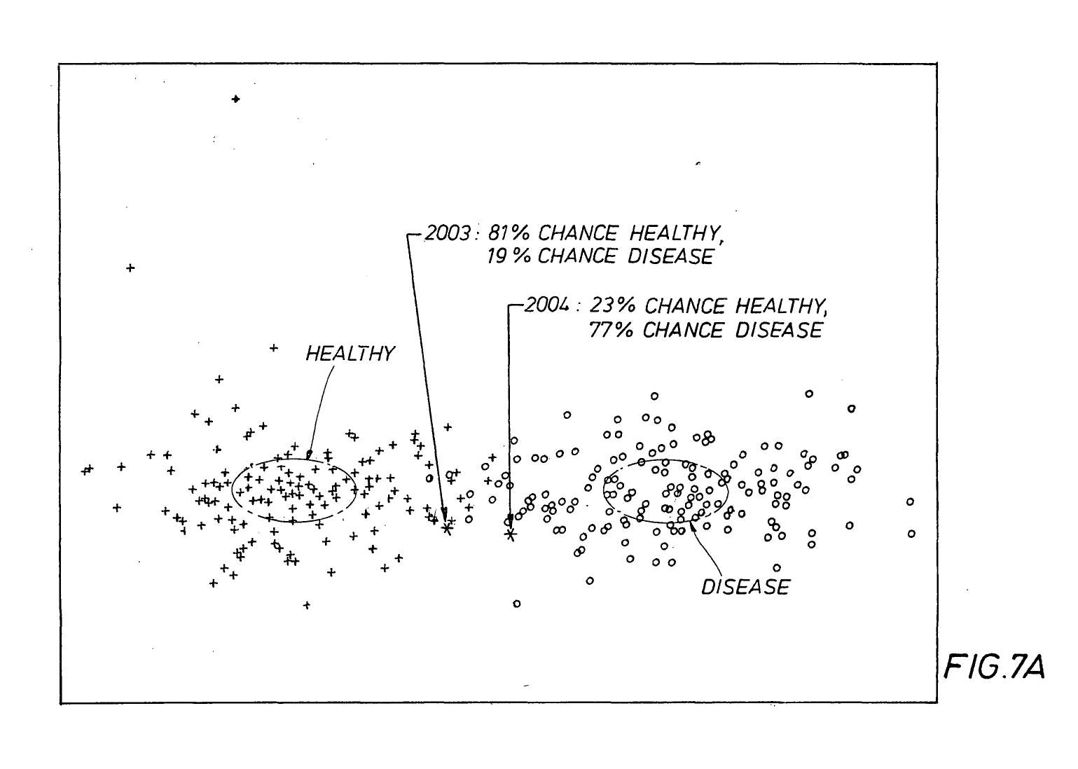

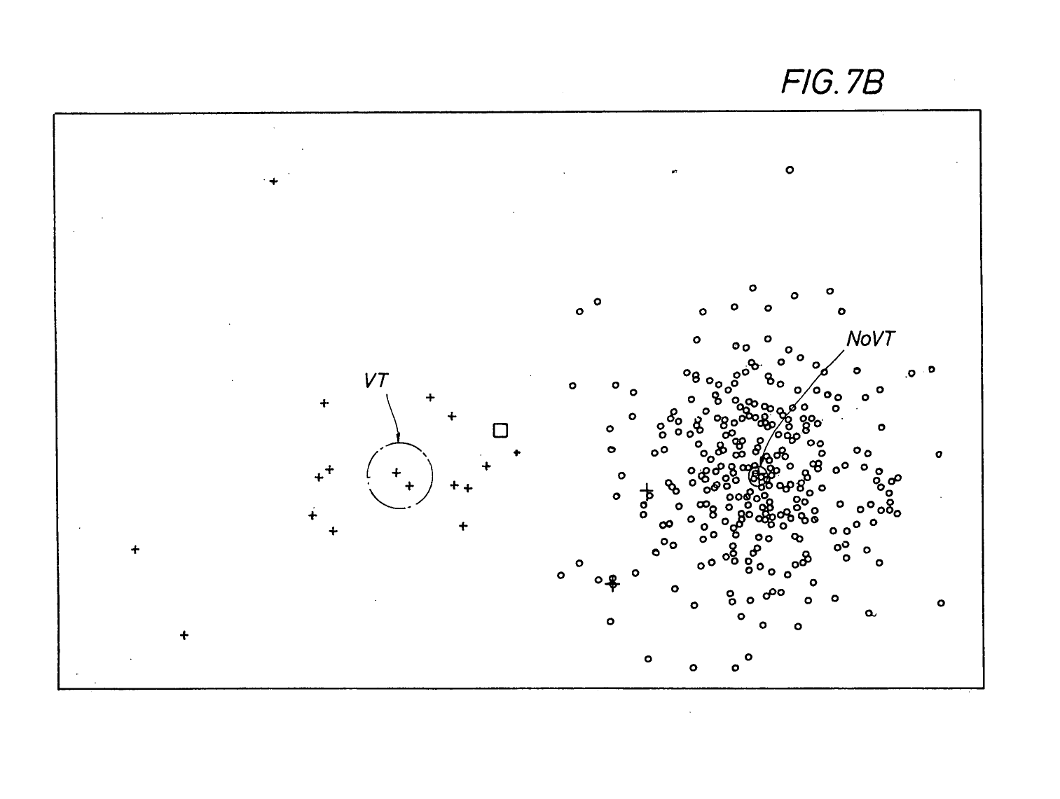

FIG. 7 shows examples of yet another aspect of the present methodology which identifies disease based on specific discriminant analysis using advanced plus conventional ECG. In the top panel, a given individual, whose data points are shown by the arrows, has been followed longitudinally over a period of one year. During that time, the individual's chance (probability) of disease by the given discriminant analysis Superscore increased from 19% to 77%. In the second panel, the specific discriminant analysis shows where individuals with a history of ventricular tachycardia or sudden cardiac death are discriminated from those who have not had these cardiac events. In this case, less than 1% of individuals are retrospectively misclassified. The 3 misclassified data points are represented by the symbols shown in bold.

The following paragraphs discuss several specific advanced ECG parameters and their deriving algorithms that, along with better known conventional ECG parameters may be utilized in the present invention.

First, there are a number of advanced ECG parameters that can be derived from Signal Averaging, with or without concomitant filtering (including digital bandpass filtering). These include a number of measures of unfiltered or filtered P, QRS or T waveform amplitudes, durations, axes, angles, slopes and velocities derived from the signal averaged P, QRS and/or T waveforms. With respect to filtered waveforms, “higher frequency” signals in any of the P, QRS or T waveforms and/or in the ST segment that are nonvisualizable and/or nonquantifiable through mere inspection of the conventional ECG tracing, due to their relatively high frequency content, are quantified by one or more computer algorithms. High Frequency P wave algorithms measure, in real-time and on a beat-to-beat basis if desired, higher frequency signals (usually >30-40 Hz) present within the P wave or within the PR interval (for example within the so-called H-V interval), preferably by employing signal averaging and digital filtering. They may be useful in helping to diagnose certain conditions (such as the Brugada syndrome, etc.) or the propensity for certain arrhythmias, especially atrial arrhythmias. High Frequency QRS wave algorithms measure, in real-time and on a beat-to-beat basis if desired, high frequency signals (usually >5 Hz, and often in the ranges of 5-250 Hz, 30-250 Hz, 40-250 Hz, or 150-250 Hz) within the QRS waves (i.e., during ventricular depolarization), preferably by employing signal averaging and digital filtering, or alternatively by measuring in the detail the upward and downward slopes of the QRS complex on a sample-point-by-sample point basis. The high frequency QRS signals may be categorized according to various quantitative and morphological criteria, including so-called “reduced amplitude zone” criteria. These algorithms are generally more useful than conventional ECG in helping to identify myocardial ischemia, coronary artery disease and cardiomyopathies, especially in middle-aged and older individuals. High Frequency QRS/ST-segment algorithms measure, in real-time and on a beat-to-beat basis if desired, high frequency signals (usually >30 Hz, most often 40-250 Hz) in the QRS wave and ST segments, preferably by employing signal averaging and filtering. These algorithms are sometimes commonly described as “late potentials” analyses. As a stand-alone technique, these analyses have modest usefulness in predicting the propensity for ventricular arrhythmias. High frequency T wave algorithms measure, in real-time and on a beat-to-beat basis as desired, high frequency signals (usually >30 Hz) present within the T-wave, preferably by employing signal averaging and digital filtering. This is a less prevalent technique, the clinical usefulness thereof as a “standalone” technique being still under evaluation.

Second, there are advanced ECG parameters of Waveform Complexity that are derived from decomposition of P, QRS, and T waveforms by techniques such as principal component analysis, independent component analysis, and singular value decomposition. These derivations preferably include signal averaging as a data processing step, but they may also be obtained without such signal averaging.

In the presently preferred embodiment, singular value decomposition (SVD) is used, in real-time and on a beat-to-beat basis if desired, to derive the detailed and otherwise non-quantifiable morphology or “energy complexity” of the P, QRS and T waveforms. Specific measures include the individual waveform eigenvalues and eigenvectors that are themselves the result of SVD, as well as those derived from several secondary mathematical formulae that incorporate one or more of these eigenvalues or eigenvectors within them. All these measures may be useful for predicting the propensity for atrial arrhythmias such as atrial fibrillation (P waveform complexity), and also for identifying CAD, CM, ion channelopathies, and the propensity for SCD and ventricular arrhythmias (P, QRS and T waveform complexity, but especially T-wave complexity).

The following are specific examples of measures of waveform complexity that are presently derived from secondary mathematical formulae after the performing SVD on eight independent channels of ECG information, SVD itself decomposing the measured set of signals (e.g., ECG channels I, II, and V1 . . . V6) into a set of the eigen (=proper) signals.

The modified Complexity Ratio (mCR) of the given P, QRS or T waveform, which is the ratio of the sum of the squares of the last six eigenvalues of the given waveform to the sum of the squares of all eight eigenvalues of the given waveform, multiplied by 100:

$$mCR = 100 \times {\sum }_{i = 3}^{8} {ρ}_{i}^{2} / {\sum }_{i = 1}^{8} {ρ}_{i}^{2}$$

$$where$$

$${ρ}_{1} \geq {ρ}_{2} \geq … \geq {ρ}_{8}$$

The Principal Component Analysis (PCA) ratio of the given P, QRS or T waveform, which is the ratio of the second to the first waveform eigenvalues, multiplied by 100:

$$PCA = 100 \times \frac{{ρ}_{2}}{{ρ}_{1}}$$

The normalized volume (nV) of the given waveform, which is the product of the second and third eigenvalues of the given waveform, divided by the square of the first eigenvalue of the given waveform (thereby yielding results for the so-called nPV, nQRSV, and nTV parameters, respectively).

On occasion, one or more individual eigenvalues is itself diagnostically more powerful (or contributory to a given Superscore) than any ratio or product or other formula involving multiple eigenvalues, such that the individual eigenvalue(s) itself is instead preferentially used in a given Superscore. For example, in our databases, the second P-wave eigenvalue is presently more powerful than any P-wave complexity ratio or product involving multiple P-wave eigenvalues, in terms of detecting cardiomyopathy.

Third. there are a number of advanced ECG parameters that together, constitute the so-called derived or reconstructed Spatial (3-dimensional) ECG. This type of advanced ECG technique employs mathematical transformations (for example, the inverse Dower or Kors' regression transformation coefficients) to transform standard 8-channel (i.e., 12-lead) or other multichannel ECG information into orthogonal (or “X, Y and Z”) components, with or without concomitant signal averaging and/or filtering. Derived spatial or “3-dimensional” ECG parameters utilized in the presently preferred embodiment of the invention include the spatial ventricular gradient time magnitude and direction (including as projected in the frontal, horizontal and sagittal planes) and its individual components (i.e., the spatial mean QRS, ST and T waves); the relationships between, as well as the beat-to-beat variation of, the spatial ventricular gradient and its components (measured stochastically or deterministically); the spatial mean QRS-T, P-QRS and P-T angles; the spatial ventricular activation time; the spatial mean P-wave time magnitude and the mean and maximum spatial velocities of the spatial P, QRS and T waves; for an individual or signal-averaged P, QRS or T waveform or ST segment, the total root mean square voltage and total integral of the derived X, Y, and Z leads either individually, or taken together as a vector magnitude, with or without bandpass filtering (e.g., 5-150 Hz, 5-250 Hz, etc.); and the so-called “derived-lead” late potentials parameters from the transformed, signal-averaged and filtered signals, including the filtered QRS duration, the RMS voltage of the terminal filtered QRS complex, and the duration of low amplitude (<40 uV) signal in the terminal QRS complex. The “spatial mean QRS-T angle” has a particularly strong predictive value for heart disease events and mortality in both the general older population and in women. It and other 3-dimensional ECG parameters are also helpful for detecting enlargement of the ventricles when the conventional ECG is falsely negative. Moreover, the spatial ventricular gradient and its variability (or that of its components) are known to be useful for detection of ischemic heart disease syndromes and ion channelopathies.

Finally, there are a number of advanced ECG parameters that can be derived from single and/or multichannel Beat-to-Beat Variability techniques, preferably but not necessarily utilizing a signal averaging component as part of the method for determining beat-to-beat variability. In the presently preferred embodiment of the invention, these measurements of beat-to-beat ECG interval variability determine, during a period that is usually at least a couple of minutes in duration, and in real-time if desired, the variability of the PP, RR, PR (PQ), QRS, and QT intervals (if desired, they can also determine the variabilities of some part of the QT interval, for example those of the Q-Tpeak, RT, R-Tpeak, JT, J-Tpeak, or Tpeak-Tend intervals). They also determine the beat-to-beat variabilities of the P, QRS and T waveform amplitudes, and other advanced parameters of variability including, for example: 1) the “unexplained” interval variability, wherein that part of the given interval's (e.g., the QT interval's) variability that can be readily explained by RR interval variability and/or by other extrinsic factors ascertainable from the advanced ECG (such as respiration-related changes in voltage amplitudes, QRS-T angles and other factors) is eliminated from total interval variability, thus isolating the variability's “unexplained” portion; and 2) ECG dipole variability utilizing for example a set of real or derived X, Y, Z dipole vectors optimally matching the eigenvectors of a singular value decomposition transformation matrix.

The variability of, for example, the QT interval from beat-to-beat is typically more sensitive than the length of the conventional QT interval itself for detecting a variety of cardiac pathologies. Specifically, an increase in QT interval variability is often more useful than is a prolongation in the conventional QT interval itself for identifying CAD and for predicting an increased propensity for life-threatening ventricular arrhythmias in individuals with pre-existing heart disease. Similarly, increases in the spatial ventricular gradient variability and in the PR interval variability may be useful for determining the presence of CAD and for predicting the propensity for atrial arrhythmias, respectively, etc.

Besides those techniques mentioned above, the results of several other advanced ECG techniques not specifically addressed above might also be easily incorporated into one or more ECG Superscores by anyone who might become skilled in the art of utilizing such advanced scores or entities in the future, according to the spirit of the invention.

Typically, for a given disease category (for example CAD) or for a given event (for example ventricular arrhythmia) there may be several specific ECG Superscores that have formulae optimized for accuracy according to the present methodology. A very specific example of one ECG Superscore that can be used to detect cardiac disease in general is shown below. This particular Superscore incorporates 14-parameters (and accompanying weighting coefficients) that were derived using a branch-and-bound parameter selection procedure within the context of a logistic regression model. Several of the parameters are also normalized via their natural logarithms (Ln):

Superscore (disease or event)=5.460264*(QT variability index in lead II)+0.0355342*(mean spatial QRS-T angle)+2.063736*(Ln nTV)+14.26611*(Ln P duration)+0.5239478*(nPV)−6.888789*(Ln Sokolow-Lyon voltage)−6.78717*(Ln normalized P Eigenvalue #2)−0.0421065*(QRS frontal plane axis)+0.1889997*(spatial ventricular gradient horizontal plane axis)+10.55984*(Ln spatial ventricular activation time)+2.586874*(Ln root mean square of the sequential differences in QT intervals in lead V2)+9.871023*(Ln alpha 2 of RR variability)+201.6318*(spatial mean QRS voltage)+2.036166*(Ln spatial P-QRS angle)-75.90054.

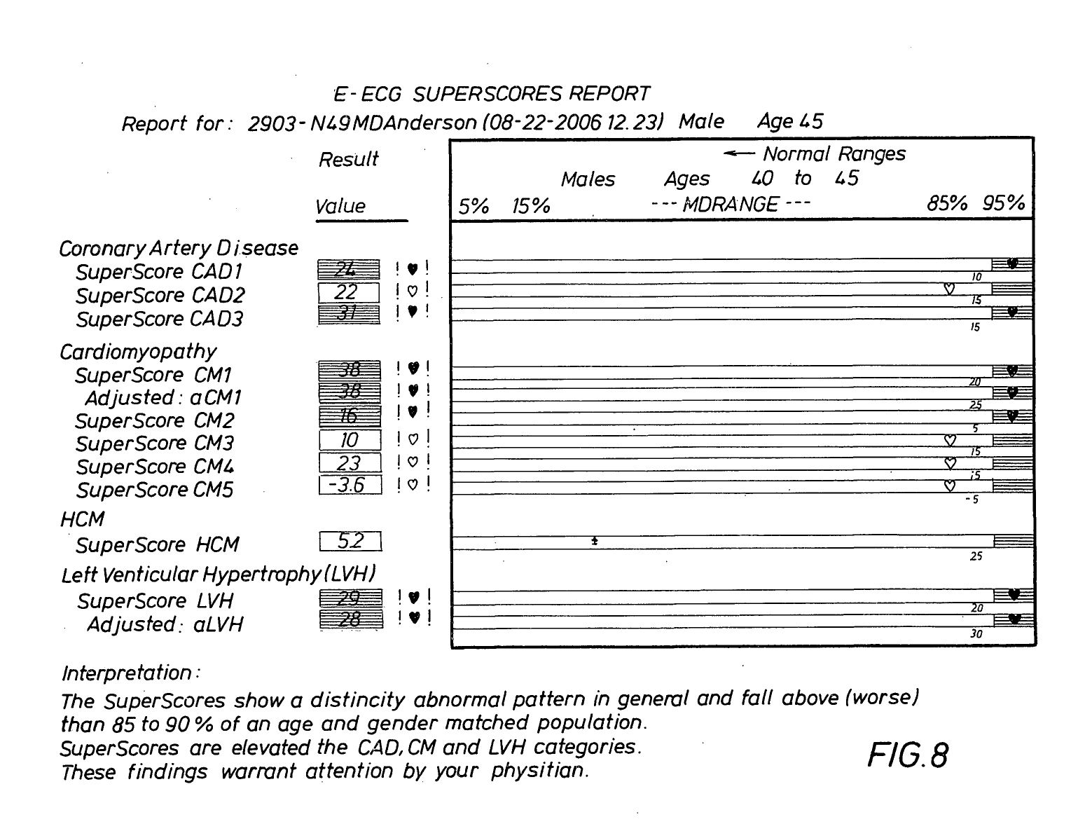

FIG. 8 illustrates a summarized computer monitor display or printout of comprehensive ECG Superscores for multiple diseases, where each has been normalized and scaled to facilitate ease of use and recognition of normal versus abnormal results. Such a display is representative of a Superscore report that may be readily utilized by a physician and/or a patient in understanding the overall Superscore results.

The principles, preferred embodiments, and mode of operation of the present invention have been described in the foregoing specification. This invention is not to be construed as limited to the particular forms disclosed, since these are regarded as illustrative rather than restrictive. Moreover, variations and changes may be made by those skilled in the art without departing from the spirit of the invention.

I claim:

1. A method of stratifying the probability of the presence and/or risk of any given cardiac disease or the risk of any given cardiac event for an individual patient comprising the steps of:
a) collecting advanced and conventional ECG data from a patient in one or more recording sessions to obtain results for a set of parameters, including for at least two parameters derived from at least two different types of advanced ECG techniques and for at least one parameter derived from the conventional ECG technique, or including for at least three parameters derived from at least three different types of advanced ECG techniques, and wherein an advanced ECG technique is defined as a technique that produces a result that a trained clinician cannot ascertain or readily calculate through visual inspection of conventional ECG tracings; and
b) combining the results of the at least three parameters from a set of parameters in an additive multivariate statistical model or pattern recognition procedure, thereby accurately assessing the probability of the given cardiac disease or the relative level of risk of the given cardiac event for the individual patient.

2. The method of claim 1, further comprising the steps of collecting, recording, and simultaneously displaying results and combinations of results from the at least three parameters from the advanced and conventional ECG techniques in real-time on a monitor, thus enabling comparison in a beat-by-beat manner or comparison otherwise over time.

3. The method of claim 1, further comprising the steps of recording and subsequently displaying combinations of the at least three parameters from the advanced and conventional ECG techniques in a graphical form.

4. The method of claim 3, wherein the graphical form comprises the results of one or more additive models, support vector machines, discriminant analyses, neural networks, recursive partitioning analyses, classification and regression tree analyses or any similar type of multivariate statistical model or pattern recognition procedure.

5. The method of claim 3 wherein graphical form comprises display on a monitor or display on a printed page.

6. A method of stratifying the probability of the presence and/or risk of any given cardiac disease or the risk of any given cardiac event for an individual patient comprising the steps of:
a) collecting advanced and conventional ECG data from a patient in one or more recording sessions to obtain results for a set of parameters, including for at least two parameters derived from at least two different types of advanced ECG techniques and for at least one parameter derived from the conventional ECG technique, or including for at least three parameters derived from at least three different types of advanced ECG techniques, and wherein the advanced ECG techniques comprise:
signal averaging of P, QRS and T waveforms, with or without accompanying bandpass filtering, to derive filtered or unfiltered parameters of waveform amplitudes, durations, axes, angles, slopes and velocities;
decomposition of P, QRS, and T waveforms, including of signal averaged P, QRS and T waveforms, by techniques such as principal component analysis, independent component analysis, and singular value decomposition, to derive not only individual eigenvalues and eigenvectors for the P, QRS and T waveforms separately or in combination, but also any number of mathematical relationships between the eigenvalues and eigenvectors of these waveforms;
spatial studies of the P, QRS and T waveforms, including of signal averaged P, QRS and T waveforms, wherein three-dimensional (e.g., X, Y, Z-channel type) ECG information is reconstructed from non-X, Y, Z-channel type systems such as the standard 12-lead or other multichannel ECG, and utilized to derive parameters such as the spatial magnitudes, durations, vector orientations, spatial angles, spatial velocities, and vector magnitudes of the unfiltered or filtered spatial P, QRS and T waveforms, the spatial angles between the unfiltered or filtered spatial P, QRS and T waveforms, and the time magnitude, angles and beat-to-beat variabilities of the unfiltered or filtered spatial angles, spatial ventricular gradient and its components;
beat-to-beat variability studies of the P, QRS and T waveforms or of the time intervals between or amongst them, including for example parameters of beat-to-beat RR, PP, PR PQ, QRS, QT, Q-Tpeak, RT, R-Tpeak, JT, or J-Tpeak variability, beat-to-beat variabilities of the P, QRS or T waveform amplitudes or of ST segment amplitudes, and other advanced parameters of variability including, for example, “unexplained” interval variability, wherein that part of the given interval's (e.g., QT interval's) variability that can be readily explained by RR interval variability and/or by other extrinsic factors ascertainable from the advanced ECG (such as respiration-related changes in voltage amplitudes, QRS-T angles and other factors) is eliminated from total interval variability, thus isolating the variability's “unexplained” portion, as well as indices of ECG dipole variability utilizing a set of real or derived X, Y, Z dipole vectors optimally matching the eigenvectors of a singular value decomposition transformation matrix;
b) combining at least two advanced ECG measurements from at least two of the different advanced ECG techniques, and including these measurements in an additive multivariate statistical model or pattern recognition procedure with at least one other advanced or conventional ECG measurement, thereby accurately assessing the probability of disease, the risk of disease, or the risk of events associated with disease for an individual patient.

7. The method of claim 6, further comprising the steps of collecting, recording and simultaneously displaying results and combinations of results from the at least three parameters from the advanced and conventional ECG techniques in real-time on a monitor, thus enabling comparison in a beat-by-beat manner or comparison otherwise over time.

8. The method of claim 6, further comprising the steps of recording and subsequently displaying combinations of the at least three parameters from the advanced and conventional ECG techniques in a graphical form.

9. The method of claim 8, wherein the graphical form comprises the results of one or more additive models, support vector machines, discriminant analyses, neural networks, recursive partitioning analyses, classification and regression tree analyses or any similar type of multivariate statistical model or pattern recognition procedure.

10. The method of claim 8 wherein the graphical form comprises display on a monitor or display on a printed page.
